(19) United States
(12) Patent Application Publication
(10) Pub. No.: US 2025/0352731 A1
(43) Pub. Date: Nov. 20, 2025

(54) MEDICATION DELIVERY DEVICES, SYSTEMS AND METHODS

(71) Applicant: BIGFOOT BIOMEDICAL, INC, Milpitas, CA (US)

(72) Inventors: Matthew James CLEMENTE, San Jose, CA (US); Rachael Elizabeth PRICE, Palo Alto, CA (US)

(21) Appl. No.: 18/871,380

(22) PCT Filed: Jun. 2, 2023
(86) PCT No.: PCT/US2023/067866

§ 371 Date: Dec. 3, 2024

Related U.S. Application Data
(60) Provisional application No. 63/365,849, filed on Jun. 3, 2022.
(60) Provisional application No. 63/382,443, filed on Nov. 4, 2022.

(51) Int. Cl. A61M 5/24; A61M 5/31

(52) U.S. Cl. CPC A61M 5/2459; A61M 2005/3126; A61M 2202/07; A61M 2205/3569; A61M 2205/505; A61M 2230/201

(57) ABSTRACT

Medication delivery methods, devices and systems are provided, the medication delivery device includes user interface elements, control circuitry, and a memory. The method includes receiving analyte measurement data of a user, presenting information related to the analyte measurement data to the user, and determining an intended dose of medication based on at least one of the analyte measurement data, optionally, therapeutic relevant conditions, therapy parameters, or a calculated insulin-on-board (IOB) value. When the user is queried to indicate that the user is ready for the delivery without presenting the intended dose on any of the one or more user interface elements of the medication delivery device, the intended dose is automatically delivered after receiving the user's indication.

### CROSS-REFERENCE TO RELATED APPLICATIONS

This application claims priority to U.S. Application Ser. Nos. 63/365,849, filed on Jun. 3, 2023 and 63/382,443, filed on Nov. 4, 2022. The disclosures of the prior applications are considered part of the disclosure of this application, and are incorporated in their entirety into this application.

### TECHNICAL FIELD

This disclosure relates generally to medication delivery devices and methods. More specifically, the disclosure relates to devices and methods for automatically delivering an intended dose of medication to a user.

### BACKGROUND

Diabetes treatment devices (e.g., glucose meters, insulin pumps, insulin injection devices, etc.) are widely used by a person with diabetes (PWD) to self-administer external biologically effective drugs (e.g., insulin or its analog). Some diabetes treatment devices or mobile applications are equipped with insulin bolus calculators to recommend insulin bolus dosages to a PWD. Typically, it is up to the patient to decide the actual amount of drug to be delivered.

### SUMMARY

This disclosure relates generally to medication delivery devices and methods. More specifically, the disclosure relates to devices and methods for automatically delivering an intended dose of medication to a user without presenting the dose to the user on any of the user interfaces of the medication delivery devices.

Briefly, in one embodiment, the present disclosure describes a method of delivering medication using a medication delivery device. The medication delivery device includes one or more user interface elements, control circuitry, and a memory. The method includes receiving, via the medication delivery device, analyte measurement data of a user; presenting, via the one or more user interface elements, information related to the analyte measurement data to the user; determining an intended dose of medication based on at least one of the analyte measurement data, optionally, one or more therapeutic relevant conditions, one or more therapy parameters stored in the memory of the medication delivery device, or a calculated insulin-on-board (IOB) value; querying the user to indicate that the user is ready for delivering medication without presenting the intended dose on any of the one or more user interface elements of the medication delivery device; and automatically delivering the intended dose after receiving the user's indication.

In another embodiment, the present disclosure describes a medication delivery device includes a housing configured to receive a medication cartridge. The medication cartridge extends between a distal end and a proximal end thereof and includes a vial having a septum at the distal end and a displacement member inside the vial and movable from the proximal end to the distal end to deliver medication received by the vial from the distal end. The medication delivery device further includes a drive mechanism inside the housing configured to advance the displacement member within the medication cartridge to deliver medication when the medication cartridge is received in the housing, and one or more location sensors positioned to detect a location of the displacement member within the medication cartridge when the medication cartridge is received in the housing. The medication delivery device further includes control circuitry configured to determine an intended dose of medication to be delivered, and obtain information from the one or more locations sensors. The information indicates the location of the displacement member within the medication cartridge. The control circuitry is further configured to determine a first position of the displacement member within the medication cartridge based on the information from the one or more location sensors. The first position identifies the location of the displacement member before delivery of the dose of medication. The control circuitry is further configured to determine a second position of the displacement member within the medication device based on the intended dose of medication to be delivered, the second position indicating a desired location of the displacement member within the medication cartridge after delivery of the dose of medication. The control circuitry is further configured to instruct the drive mechanism to advance the displacement member from the first position, and instruct the drive mechanism to cease advancing the displacement member upon obtaining the information from the one or more location sensors indicating that the displacement member has reached the second position.

In another embodiment, the present disclosure describes a method of delivering medication using a medication delivery device that includes a housing configured to receive a medication cartridge, the medication cartridge extending between a distal end and a proximal end thereof and including a vial having a septum at the distal end and a displacement member inside the vial and movable from the proximal end to the distal end to deliver medication received by the vial from the distal end, the medication delivery device further including a drive mechanism inside the housing configured to advance the displacement member within the medication cartridge to deliver medication when the medication cartridge is received in the housing, one or more location sensors positioned to detect a location of the displacement member within the medication cartridge, and control circuitry configured to control operation of the medication delivery device, the method including the control circuitry determining an intended dose of medication to be delivered; the one or more location sensors detecting a location of the displacement member within the medication cartridge and providing information indicating the location of the medication cartridge to the control circuitry; the control circuitry obtaining the information from the one or more locations sensors indicating the location of the displacement member within the medication cartridge; the control circuitry determining a first position of the displacement member within the medication cartridge based on the information from the one or more location sensors, wherein the first position identifies the location of the displacement member before delivery of the dose of medication; the control circuitry determining a second position of the displacement member within the medication device based on the intended dose of medication to be delivered, the second position indicating a desired location of the displacement member within the medication cartridge after delivery of the dose of medication; the control circuitry instructing the drive mechanism to advance the displacement member from the first position; and the control circuitry instructing the drive mechanism to cease advancing the displacement member upon obtaining the information from the one or more location sensors indicating that the displacement member has reached the second position.

Various aspects and advantages of exemplary embodiments of the disclosure have been summarized. The above Summary is not intended to describe each illustrated embodiment. Other features and aspects will become apparent by consideration of the following detailed description and accompanying drawings.

### BRIEF DESCRIPTION OF THE DRAWINGS

References are made to the accompanying drawings that form a part of this disclosure and which illustrate the embodiments in which systems and methods described in this specification can be practiced.

FIG. 1 is a block diagram of a medication delivery and management system, according to one embodiment.

FIG. 2 is a block diagram of a medication delivery device, according to one embodiment.

FIG. 3 is a top perspective view of a medication delivery device, according to one embodiment.

FIG. 4A is a side perspective view of the medication delivery device of FIG. 3.

FIG. 4B is a first end view of the medication delivery device of FIG. 4A.

FIG. 4C is a second end view of the medication delivery device of FIG. 4A.

FIG. 5A is a cross-sectional view of the medication delivery device of FIG. 3.

FIG. 5B is a cross-sectional view of a portion of the medication delivery device of FIG. 5A.

FIG. 6 is a flow diagram of a method of delivering medication using a medication delivery device, according to one embodiment.

FIG. 7 is a flow diagram of a method of delivering medication using a medication delivery device, according to another embodiment.

FIG. 8 illustrates screenshots of a user interface of a medication delivery device, according to some embodiments.

Particular embodiments of the present disclosure are described herein with reference to the accompanying drawings; however, it is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. In this description, as well as in the drawings, like-referenced numbers represent like elements that may perform the same, similar, or equivalent functions.

### DETAILED DESCRIPTION

This disclosure relates generally to medication delivery devices and methods. More specifically, the disclosure relates to devices and methods for automatically delivering an intended dose of medication to a user, in particular, without presenting any dose information to the user on any user interface elements of the medication delivery device.

Systems, devices and methods described herein can be applied to deliver various medications and manage various diseases including, for example, diabetes, blood thinners such as Heparin, palliative care pain medications, growth hormone deficiency treatment such as somatotropin, fertility treatment such as human chorionic gonadotropin or follicle stimulating hormone, etc.

In some embodiments, systems, devices and methods described herein can provide multiple, daily delivery of medication, for example, a mixture of rapid-and long-acting insulin via a medication delivery device to a user. The systems, devices and methods allow the user (e.g., a person with diabetes or PWD) to easily administer the multiple, daily delivery of medication without burdening the user with the mental task to calculate the appropriate medication bolus dosage, to evaluate the effect of therapeutic relevant conditions (e.g., meal sizes, exercise plans, or other lifestyle factors) on the medication bolus dosage, to determine the actual doses that have been delivered, etc.

### Medication Delivery and Management System

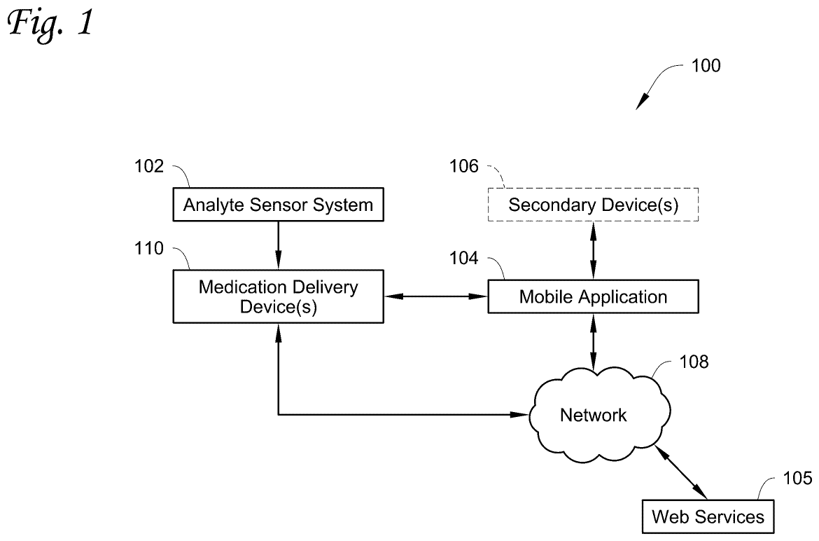

FIG. 1 illustrates a medication delivery and management system 100. In one embodiment, the system 100 is an insulin therapy delivery and management system. It is to be understood that the system 100 can be applied to deliver various medications and manage various diseases. The system 100 includes an analyte sensor system 102, one or more medication delivery device(s) 110, a mobile application 104, and optionally, one or more secondary device(s) 106. The medication delivery and management system 100 may further include one or more web services 105 that communicate with the mobile application 104 by way of a network 108 (e.g., a cloud networking). The one or more medication delivery device(s) 110 may join and leave the system 100, interact with users to determine an intended dose, and automatically deliver the intended dose of medication to the users without presenting the intended dose on any user interface element of the medication delivery device(s) 110.

In some cases, the one or more medication delivery device(s) 110 can be an injector that might have a suitable shape such as, for example, a pen shape. In some cases, the one or more medication delivery device(s) 110 can include an insulin delivery device such as, for example, an insulin injection pen. In some cases, the insulin injection pen can be reusable by accepting an insulin cartridge, ejecting it, and accepting a new insulin cartridge.

The analyte sensor system 102 is configured to obtain various analyte measurement data and communicate the obtained analyte measurement data to the medication delivery device(s) 110. In some cases, the analyte sensor system 102 may include a glucose sensor system adapted to determine glucose values including, for example, a blood glucose meter (BGM), a flash glucose monitor, a continuous glucose monitor (CGM), etc. In some cases, the glucose sensor system 102 can act as a flash glucose monitor, a continuous glucose monitor, or both by permitting intermittent and/or on-demand transmissions of glucose data. In some cases, the glucose sensor system 102 may monitor the change of glucose values in a given period of time and provide glucose trend data. In some cases, the analyte sensor system 102 can wirelessly transmit data when interrogated by a reader device (e.g., using NFC communication). In some cases, the glucose sensor can wirelessly transmit data at predetermined intervals (e.g., using radio frequencies) using any suitable communication standard (e.g., Bluetooth Low Energy (BLE)). In some cases, the analyte sensor system 102 may include multiple glucose sensor systems (e.g., one or more of a continuous glucose monitor, a flash glucose monitor, a glucose meter, etc.).

The analyte sensor system 102 can transmit analyte measurement data (e.g., glucose data) using multiple communication techniques. In some embodiments, the mobile application 104 and/or one or more of the medication delivery device(s) 110 may include an NFC reader adapted to obtain analyte measurement data from the analyte sensor system 102 when brought within an interrogation distance of the analyte sensor system 102. In some embodiments, the mobile application 104 and/or one or more of the medication delivery device(s) 110 may wirelessly receive analyte measurement data from the analyte sensor system 102 that is broadcast at predetermined periods of time (e.g., every 30 seconds, every minute, every 2 minutes, every 3 minutes, every 5 minutes, every 10 minutes, every 15 minutes, etc.).

In an interrogated mode of operation, the analyte sensor system 102 may wirelessly send analyte measurement data to the mobile application 104 and/or one or more of the medication delivery device(s) 110. For example, when the medication delivery device(s) 110 interrogates the glucose sensor system 102, it may receive stored glucose data from the previous 1 hour, 2 hours, 3, hours, 4 hours, 5 hours, 6 hours, 7 hours, 8 hours, etc. In some cases, broadcast glucose data may include a current or more recent glucose value and current glucose trend data. In some cases, the mobile application 104 and/or one or more of the medication delivery device(s) 110 may receive the most current glucose data (e.g., from the last 5, 10, 20 or 30 minutes) directly from the glucose sensor system 102, and issue alarms, alerts or notifications based on the most current glucose data.

The medication delivery device(s) 110 can be configured to capture information related to the delivery of medication, including, for example, one or more user interface elements for presenting or displaying information and receiving user input, internal sensors for actual dose capture, and other communication interfaces for wireless or wired communication with one or more of the other medication delivery device(s) 110, the analyte sensor system 102, the mobile application 104, and the optional secondary device(s) 106. In some cases, the medication delivery device(s) 110 may be associated with various particular types of insulin such as, for example, a rapid-acting insulin, a long-acting insulin, a mixture of rapid-acting and long-acting insulin, etc. A rapid-acting insulin may begin to work about 15 minutes after injection, peak in about one or two hours after injection, and last between about two to four hours. A long-acting insulin may reach the bloodstream several hours after injection and tend to lower glucose levels up to 24 hours. In some embodiments, a first medication delivery device 110 can be associated with long-acting insulin delivery, and a second medication delivery device 110 can be associated with rapid-acting insulin delivery. It is to be understood that the medication delivery device(s) 110 may be configured to deliver any types of insulin other than a rapid-acting insulin and a long-acting insulin.

The medication delivery device(s) 110 can execute one or more algorithms to determine an intended dose of medication based on analyte measurement data from the analyte sensor system 102. In some cases, the intended dose can be determined further based on one or more therapeutic relevant conditions from a user's input, one or more therapy parameters stored in a memory of the medication delivery device(s) 110, or a calculated insulin-on-board (IOB) value. The medication delivery device(s) 110 can have one or more user interface elements to present a query to the user to indicate that the user is ready for the delivery without presenting the intended dose on any of the user interface elements of the medication delivery device(s) 110. After receiving the user's indication, the medication delivery device(s) 110 can automatically deliver the intended dose.

In various embodiments described herein, the determined intended dose is not normally displayed, via the one or more user interface elements, to a user under a normal functioning mode of the medication delivery device(s) 110. In some cases, the medication delivery device(s) may work under a service/maintenance mode where the intended dose or other dose-related information may be presented or displayed, via one of the user interface elements of the medication delivery device(s) 110, for example, to a technician for device service/maintenance. The service/maintenance mode can be triggered, for example, after an entry of a complex function on the medication delivery device(s) 110, or by a mobile application through a secondary device. In some cases, the intended dose or other dose-related information may be presented or displayed, via one or more of the user interface elements of the medication delivery device(s) 110 under certain service/maintenance modes with certain limitations, such as, for example, as small font sizes that are not readily apparent to a user.

In some embodiments, when the user intends to access and/or adjust the intended dose determined by the medication delivery device(s) 110 and not presented on any of the user interface elements of the medication delivery device(s) 110, the user may use one or more optional secondary device(s) 106 to access and/or adjust the intended dose. The secondary device(s) 106 may include, for example, a smart phone, a laptop, a tablet, a portable computer, etc. The secondary device(s) 106 each may include a user interface to interact with the user. The secondary device(s) 106 can present a query to the user to indicate whether the user needs to access and/or manually adjust the intended dose determined by the medication delivery device(s) 110. The secondary device(s) 106 can receive a user input dose representing a dose amount or an adjustment of the intended dose. The secondary device(s) 106 can be in wireless communication with the medication delivery device(s) 110 to transmit the received user input dose to the medication delivery device(s) 110. After receiving the user input dose from the secondary device(s) 106, the medication delivery device(s) 110 can present (e.g., display), via at least one of its user interface elements, an indication that the medication delivery device(s) 110 has received the user input dose from the secondary device(s) 106. The medication delivery device(s) 110 can query the user to indicate the user is ready for the delivery of the user input dose, and automatically deliver the user input dose after receiving the user's indication.

In some embodiments, the medication delivery device(s) 110 can execute one or more algorithms to determine an actual dose of medication after delivering the intended dose. The medication delivery device(s) 110 can compare the actual dose to the intended dose. When the actual dose is not the same as the intended dose, the medication delivery device(s) 110 can calculate an insulin-on-board (IOB) value based on the actual dose, and calculate a subsequent intended dose for a next predetermined time based on the calculated IOB value. The IOB value refers to the amount of active insulin remaining in the user. Based on the historical and current data of the actual dose delivered to the user, the medication delivery device(s) 110 can execute one or more algorithms to calculate or predict the current IOB value for the user.

In some embodiments, the medication delivery device(s) 110 can execute one or more algorithms to generate a query and present the query, via one or more user interface elements of the medication delivery device(s) 110, to a user to solicit the user to input therapeutic relevant conditions. The therapeutic relevant conditions may include any external factors that may biologically affect the user's demand of medication, including, for example, at least one of a meal size, an exercise plan, a sleep plan, other lifestyle factors, etc. For example, when a user intends to dose for a meal, a query may be presented, via a user interface of the medication delivery device(s) 110 to the user to solicit the user to input his indication of a meal size or category (e.g., small meal, medium meal, or large meal). The indication of the meal size or category (e.g., meal icons or indicators) may be based on a size of an icon, a displayed number of carbohydrates, a label (e.g., Small or S, Medium or M, Large or L), or other characteristics of the meals, such as, for example, preferred meal selections made by the user, meals having selected nutritional characteristics (e.g., carbohydrates), certain meals based on time of day (e.g., breakfast, lunch, dinner, snack), etc. It is to be understood that in some embodiments, the medication delivery device(s) 110 may not present, on any of its user interface elements, dose recommendations for different meal sizes having a small, medium, or large impact on blood sugar. Instead, the medication delivery device(s) 110 receive the user's input of the one or more therapeutic relevant conditions (e.g., a meal size), and determine an intended dose of medication based on the received therapeutic relevant conditions without presenting the dose on any of the user interface elements of the medication delivery devices.

In some embodiments, the query to the user of the therapeutic relevant conditions may be generated or determined based on personalization pattern data of the user. The personalization pattern data of a user may include the user's lifestyle factor data or historical data relating to, for example, user meal time in a day, user medication delivery time in a day, user exercise time in a day, user sleep time in a day, analyte measurement data (e.g., glucose level) in response to the user's input of different therapeutic relevant conditions, actual dose data, etc. The lifestyle factor data or the historical data may be collected by the analyte sensor system 102, the medication delivery device(s) 110, the mobile application 104, and/or other components of the system 100. The medication delivery device(s) 110 can include memory that stores the user's personalization pattern data. The medication delivery device(s) 110 can determine the content of the query and the timing (e.g., time-of-day) to present the query to a user on its user interface elements based on the user's personalization pattern data.

In some embodiments, the medication delivery device(s) 110 can include memory that stores user-specific therapy or dosage parameters including, for example, a predetermined daily dose of long-acting insulin or total daily basal dose (TDBD), an insulin sensitivity factor (ISF), a carbohydrate-to-insulin ratio (CR), correction amounts based on glucose level ranges, total daily insulin doses (TDD), target glucose values, recommended rapid-acting doses for different meal sizes or categories, etc. In some embodiments, the user-specific therapy or dosage parameters may be time or day dependent. For example, CR and ISF values may depend on the hour of the day. In some embodiments, the medication delivery device(s) 110 can include memory that stores pre-determined doses of rapid-acting insulin for different meals or for different meal sizes or categories.

In some embodiments, user-specific dosage parameters and/or different pre-determined doses for different meals may be updated via the mobile application 104 in wireless communication with one or more of the medication delivery device(s) 110. For example, an algorithm in the secondary device(s) 106 or in the network 108 can update the user-specific therapy or dosage parameters. In some embodiments, the user-specific therapy or dosage parameters may be updated by a healthcare professional or manually by a user or a caregiver. In some embodiments, the medication delivery device(s) 110 can include an algorithm in memory to be executed by a processor to automatically update the user-specific therapy or dosage parameters.

The medication delivery device(s) 110 can, in some embodiments, display or otherwise provide notice to a user of a current glucose level and/or glucose trend data (e.g., a rate of glucose level change) based on glucose data received from the analyte sensor system 102. The medication delivery device(s) 110 may automatically determine an intended dose of medication (e.g., insulin) based on one or more of glucose data, user-specific therapy or dosage parameters, the user input of therapeutic relevant conditions, recommended dosage amounts set by a user or healthcare professional, time-of-day, meal data or categorizations, or any other suitable input. The mobile application 104 may be stored and executed on any suitable mobile computing device such as, for example, one or more of the secondary device(s) 106 which may be, for example, a smart phone, a laptop, a tablet, a portable computer, etc. The mobile application 104 can be adapted to input and output (e.g., display) therapy relevant information wirelessly received from the other components of the system 100. The mobile application 104 may allow a graphical user interface (GUI) that enables users to interact with the mobile application 104.

In some embodiments, the secondary device(s) 106 can store and execute a trusted mobile application within a trusted execution environment (hardware and/or software). Various functions and calculations that relate to the medication delivery and therapy management system 100, including notifications, alerts, queries, and recommendations that are presented to users may be, at least in part, performed by the trusted mobile application. In addition, some or all communication with the medication delivery device(s) 110 may be restricted to the trusted mobile application or trusted mobile computing devices. In some cases, the mobile application 104 may allow an authorized user to adjust an intended dose to be delivered by the one or more medication delivery device(s) 110 via the trusted mobile computing devices such as the secondary device(s) 106 of FIG. 1.

Generally, the embodiments of the disclosure may use any suitable wireless communication protocol for communication among the medication delivery device(s), the analyte sensor system, the secondary device(s), other electronic devices, and/or other mobile devices of the system 100. Examples of suitable wireless communication protocols include near-field-communication (ISO/IEC 14443 and 18092 compliant technology), wireless modems and routers (IEEE 802.11 compliant technology), and Bluetooth®/Bluetooth Low Energy (BLE) (IEEE 802.15 compliant technology).

In some cases, the one or more medication delivery device(s) 110 may include one or more accessories such as, for example, a cap and/or a carrying case for the medication delivery device(s) 110. The one or more medication delivery device(s) 110 and the accessories may be integrated (e.g., attached or functionally connected) and performed in an electronics package (e.g., a smart electronics).

In some cases, the one or more medication delivery device(s) 110 may be paired with the system 100 via a profile created by the mobile application 104. In one embodiment, the mobile application 104 may query web services 105 for whether a profile for a device already exists for a specific user, and, if it does, request that it be sent. The user profile may include a user's personalization pattern data including, for example, determination algorithms to determine an intended dose for the user, historical data or physiological attributes of the user (e.g., insulin sensitivity), historical or current data related to actual glucose measurements and glucose response analysis, etc. Upon creating the user profile, the mobile application 104 may save insulin therapy related settings with the profile. The insulin therapy related settings may include user-specific dosage parameters for a user, delivery characteristics of the device, specific techniques that may be used to determine an intended dose, etc. In one embodiment, each delivery device profile may include, or be part of a user profile that includes, pre-configured correction doses for particular analyte measurement data (e.g., particular glucose ranges), or for particular therapeutic relevant conditions (e.g., different meal sizes). In one embodiment, the pre-configured doses may be entered at the mobile application 104. In another embodiment the pre-configured doses may be entered at one of the web services 105 (e.g., by a healthcare provider or parent), and downloaded to the mobile application 104.

In some embodiments, the user profile including a user's personalization pattern data may be generated by the system 100 and updated over time. The system 100 may allow a user or healthcare professional, via the mobile application 104 or the web services 105, to generate the user profile, for example, by setting the user's therapy parameters (e.g., a daily dosage of long-acting insulin, dosages for different meal sizes or categories, an insulin sensitivity factor, a carbohydrate-to-insulin ratio, etc.), by creating a table of correction doses to be used for a particular range of glucose values, etc. The user profile can be stored in the memory of the medication delivery device(s) 110 and updated over time. For example, in some embodiments, one or more of the glucose data received from the analyte sensor system 102, the actual dose data determined by the medication delivery device(s) 110, or the calculated IOB values, may be analyzed to make adjustments to a user's dosage parameters and/or the meal-based dosage corrections.

### Medication Delivery Device

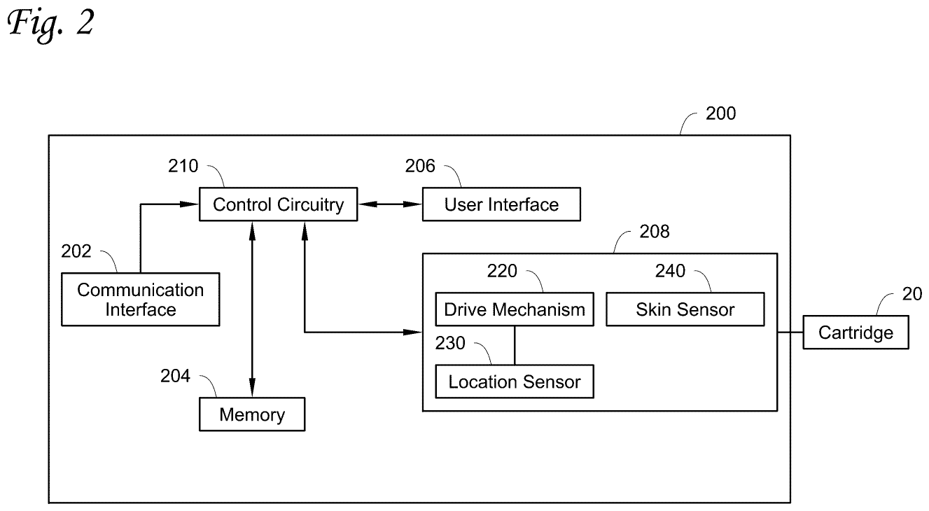

FIG. 2 is a block diagram of a medication delivery device 200 suitable for use as the medication delivery device(s) 110 in the system 100 of FIG. 1, according to some embodiments. The medication delivery device 200 includes control circuitry 210, a communication interface 202, memory 204, sensing and actuation components 208, and one or more user interface elements 206. The sensing and actuation components 208 include a drive mechanism 220, one or more location sensors 230, and one or more skin sensors 240. The drive mechanism 220 is configured to advance a displacement member (e.g., a plunger) of a medication cartridge received in the housing to deliver medication from the medication cartridge. The one or more location sensors 230 are positioned to detect the location of the displacement member (e.g., a plunger) within the medication cartridge. In some cases, the drive mechanism 220 may include an advancing mechanism (e.g., a piston) engaging with the displacement member (e.g., a plunger) of the medication cartridge. The location sensors 230 are positioned to detect the location of the advancing mechanism or the displacement member within the medication cartridge when the medication cartridge is received by the medication delivery device 200. Examples for detecting the location of the displacement member are described in U.S. Pat. Nos. 9255830; 10255991; and 10183120; and in PCT Publication Nos. WO 2019/123257; WO 2019/123258; WO 2017/009724; WO 2019/186261; WO 2020/255085; and WO 2019/123441, which are all incorporated herein by reference in their entirety. The one or more skin sensors 240 are positioned at a distal end of the housing to detect a user's skin. The control circuitry 210 receives sensing data from the skin sensors 240 to determine whether the medication delivery device 200 is at the right position, location, or orientation for medication delivery. In some cases, the various device components can be received or supported by a housing and integrated as a smart electronics device.

The communications interface 202 may include any suitable hardware, circuitry, logic, firmware and other related components of the medication delivery device 200 that are coupled to the control circuitry 210 and configured to support communications between the medication delivery device 200 and one or more external electronic devices, mobile computing devices, and a cloud networking such as network 108 shown in the system 100 of FIG. 1. In some cases, the communication interface 202 may use one or more wireless communication techniques such as, for example, near-field communication (NFC), Bluetooth low energy (BLE), WiFi, etc. In some embodiments, the communications interface 202 may also support wired communications.

The one or more user interface elements 206 may include any suitable hardware, circuitry, logic, firmware and other related components of the medication delivery device 200 configured to support user communications and interactions between the medication delivery device 200 and a user. The one or more user interface elements 206 associated with the medication delivery device 200 may include one or more output elements including, for example, a visual output device such as a display, an audio output device such as a speaker, a vibration device, etc. The one or more user interface elements 206 may further include one or more input elements, such as, for example, a button, a knob, a touch screen or panel, etc. In some cases, the one or more user interface elements 206 may include a delivery button that is actuatable by a user to trigger the drive mechanism 220 to deliver an intended dose of medication via the medication cartridge 20. In some cases, the one or more user interface elements 206 may include an ejection button that is actuatable by a user to trigger an ejection mechanism to eject the medication cartridge 20 out of the housing. A suitable delivery button or an ejection button may be a tactile depressible button that is functionally coupled with the control circuitry 210 to send corresponding actuation signals to the control circuitry 210 upon the user's different actions.

The control circuitry 210 is coupled to the communication interface 202, the memory 204, the sensing and actuation components 208, and the one or more user interface elements 206. The control circuitry 210 is configured to support the operations, tasks, and/or processes of the medication delivery devices described herein. For example, the control circuitry 210 supports operation of the sensing and actuation components 208 in response to a user's instruction from the one or more user interface elements 206 to deliver a predetermined dose of medication to the user via the medication cartridge 20 with a needle attached. Depending on the embodiment, the control circuitry 210 may be implemented or realized with a general purpose processor, a microprocessor, a controller, a microcontroller, a state machine, a content addressable memory, an application specific integrated circuit, a field programmable gate array, any suitable programmable logic device, discrete gate or transistor logic, discrete hardware components, or any combination thereof, designed to perform the functions described herein. In this regard, the steps of a method or algorithm described in connection with the embodiments disclosed herein may be embodied directly in hardware, in firmware, in a software module executed by the control circuitry 210, or in any practical combination thereof.

In various embodiments, the control circuitry 210 may further include or have accesses to the memory 204, which may include any suitable non-transitory computer-readable medium capable of storing programming instructions for execution by the control circuitry 210. The computer-executable programming instructions, when read and executed by the control circuitry 210, cause the control circuitry 210 to perform the tasks, operations, functions, and processes described herein.

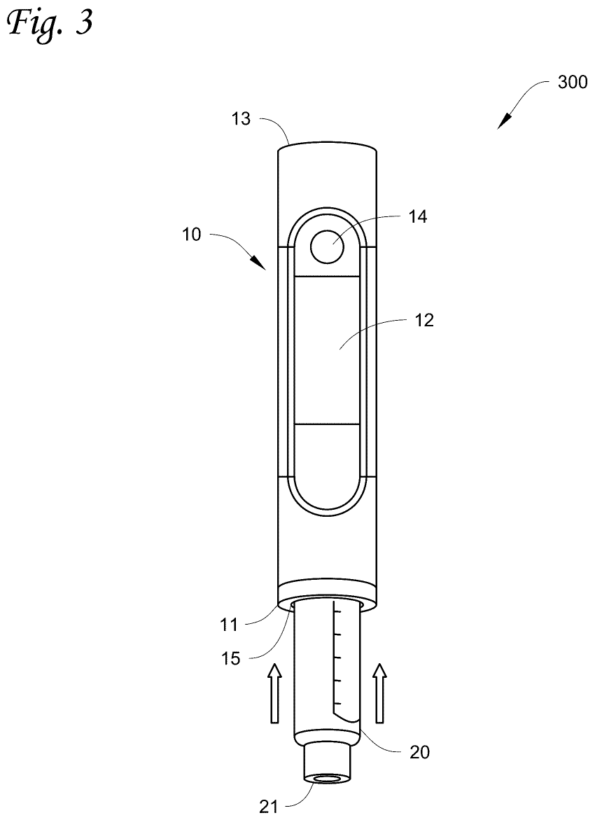

Various embodiments of a medication delivery device are provided for the medication delivery device(s) 110 of FIG. 1 and the medication delivery device 200 of FIG. 2. FIG. 3 is a perspective view of a medication delivery device 300, according to one embodiment. The medication delivery device 300 includes a housing 10 extending between a first end 11 and a second end 13 thereof in a longitudinal direction. The housing 10 defines an internal space to receive a medication cartridge 20 via an opening 15 at the first end 11 of the housing 10. As shown in FIG. 3, the medication cartridge 20 is inserted into the housing 10 via the opening 15 at the first end 11 of the housing 10 along the longitudinal direction as indicated by the arrows. In some cases, the whole body of the medication cartridge 20 can be positioned inside the housing 10, with the distal end 21 of the medication cartridge 20 being accessible from the opening 15 for a user to install a needle thereon. In some cases, a major body portion of the medication cartridge 20 can be positioned inside the housing 10, with the distal end 21 projecting outside of housing 10 through the opening 15. In the embodiment depicted in FIG. 3, the housing 10 can have a one-piece construction which can be formed of multiple pieces assembled together. In some embodiments, the housing 10 may have a first part at the first end 11 and a second part at the second end 13, and the first part and the second part can engage with each other by an engagement mechanism (e.g., a screwing mechanism). The medication cartridge can be loaded into the first and/or second part and ejected out via an ejection mechanism such as, for example, a spring loaded bayonet mechanism.

The medication delivery device 300 further includes a display panel 12 disposed on an outer surface of the housing 10. The display panel 12 may include a display to present various information to a user, including, for example, analyte measurement data (e.g., continuous glucose monitor (CGM) data, flash glucose monitor data, glucose meter data, etc.), a query to a user to solicit the user to input therapeutic relevant conditions, indicators or icons representing therapeutic relevant conditions (e.g., meal sizes, exercise plans, etc.), an alert for a glucose measurement value out of a predetermined range, a query to indicate whether the user needs to manually adjust the intended dose, an indication that the medication delivery device has received the user input dose from the secondary device, an instruction to the user to prime the medication delivery device before automatically delivering the intended dose, etc.

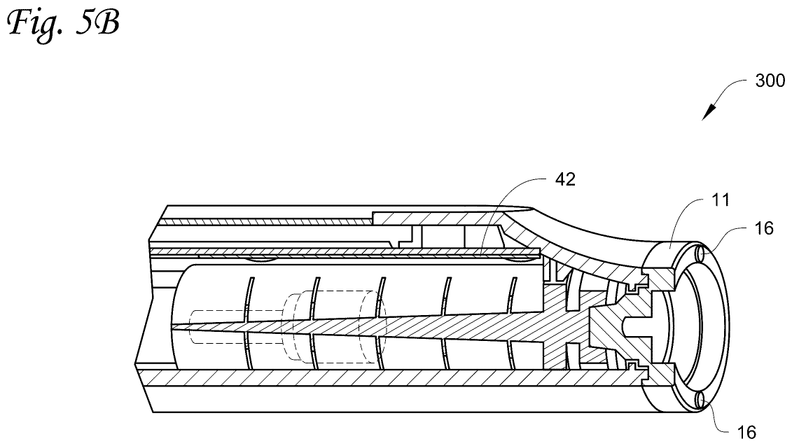

The display panel 12 can be a part of user interface (UI) of the medication delivery device 300 for the user to interact and communicate with the medication delivery device 300. The display panel 12 may further include a touch screen, one or more touch buttons, one or more smart sensors to detect a user's action (e.g., a finger touch), or other input mechanisms to receive the user's input. The one or more touch buttons on a touch screen may include, for example, an injection button, an ejection button. It is to be understood that one or more of the touch buttons on a touch screen may be a tactile button disposed on an outer surface of the housing 10. For example, in the embodiment depicted in FIG. 5A, the medication delivery device 300 includes an injection button 14 and an ejection button 18 disposed on an outer surface of the housing 10.

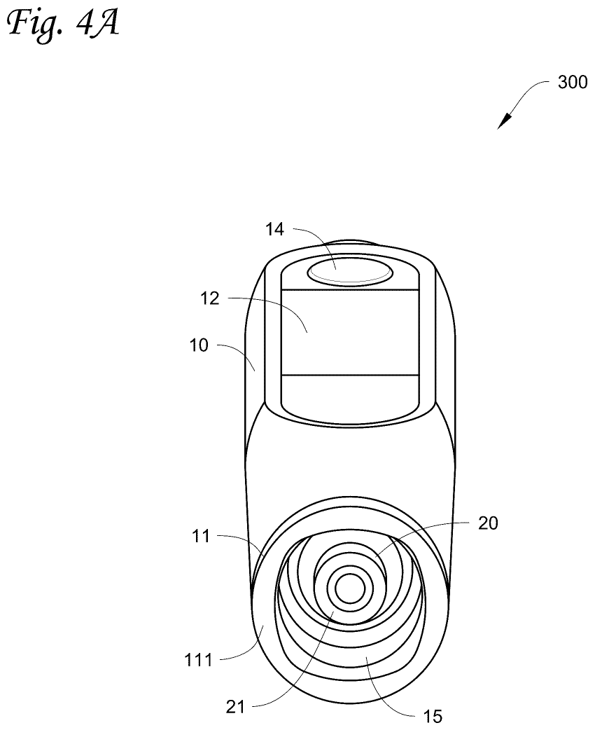

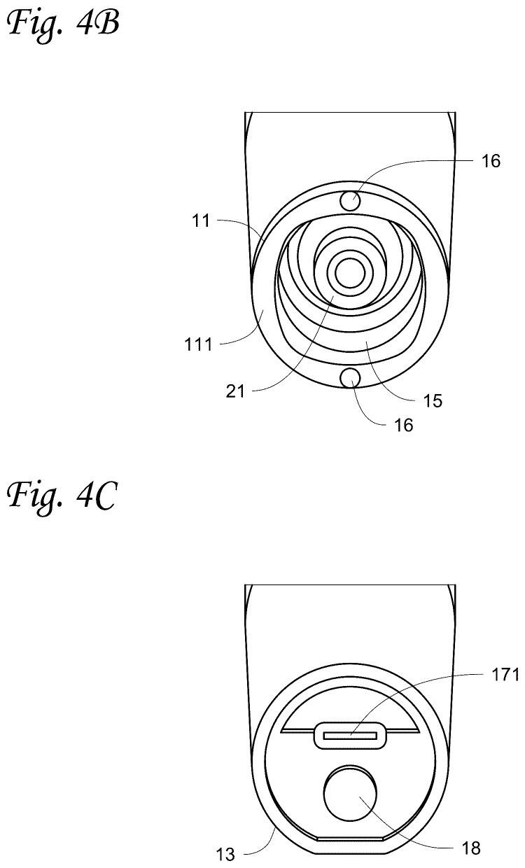

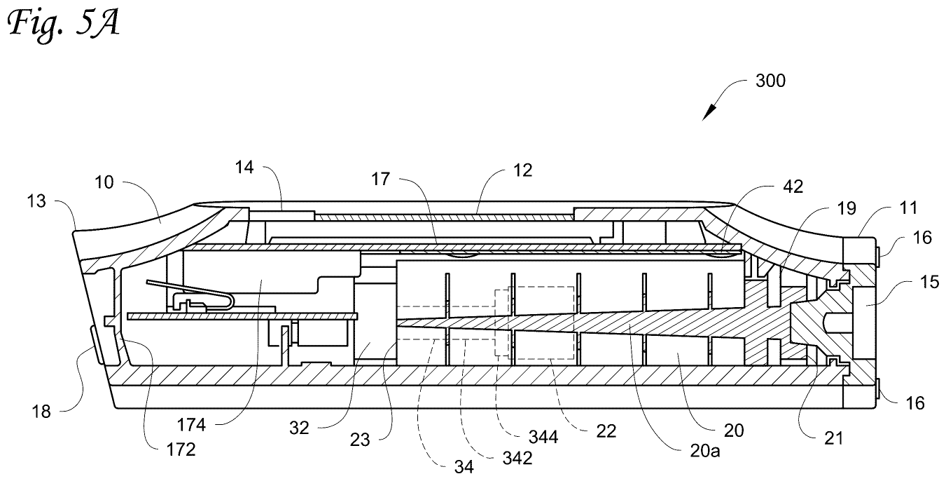

FIG. 4A is a side perspective view of the medication delivery device 300. FIG. 4B is a first end view of the medication delivery device 300. FIG. 4C is a second end view of the medication delivery device 300. As shown in FIG. 4A, the whole body of the medication cartridge 20 is positioned inside the housing 10, with the distal end 21 being indented from an edge surface 111 at the first end 11 of the housing 10. The distal end 21 is accessible from the opening 15 for a user to install a needle thereon. It is to be understood that the housing 10 can be configured to receive cartridges with different lengths.

As shown in FIG. 4B, the medication delivery device 300 includes one or more skin sensors 16 disposed on the edge surface 111 at the first end 11 of the housing 10. The skin sensors 16 are configured to detect a user's skin when the edge surface 111 at the first end 11 of the housing 10 is in close contact with the user's skin. In some cases, the skin sensors 16 may include one or more capacitive sensors disposed on the edge surface 111 and configured to measure information arising from a proximity interaction between the edge surface 111 and the user's skin. While a pair of skin sensors are illustrated in FIG. 4B, it is to be understood that one or more skin sensors 16 can be located at the first end 11 of the housing to detect the proximity of skin. In some cases, sensing data from multiple skin sensors may be used to detect the orientation of the housing 10 with respect to the user's skin. For example, when the housing 10 is tilted with an angle such that a needle is not straight into the skin, the skin sensors 16 may sense the angle and send out a signal indicating that the orientation needs to be adjusted. When the edge surface 111 and the user's skin are in an intimate contact with each other and the orientation is in an acceptable position, the skin sensors 16 may send a signal to indicate that the medication delivery device 300 is at an acceptable position for medication delivery. In some embodiments, the medication delivery device 300 may further include one or more accelerometers to determine an angle of insertion of the needle with respect to the user's skin surface.

As shown in FIG. 4C, the medication delivery device 300 includes an ejection button 18 at the second end 13 to allow a user to push to disengage and eject the medication cartridge 20 from the medication delivery device 300. A charging port 172 is also provided to allow a user to electrically charge a battery (e.g., a battery 174 as show in FIG. 5A) contained inside the housing 10. The charging port 172 may have battery charging specifications according to various industry standards (e.g., Universal Serial Bus (USB) industry standards).

FIG. 5A is a cross-sectional view of the medication delivery device 300. FIG. 5B is a cross-sectional view of a portion of the medication delivery device 300 of FIG. 5A. The housing 10 of the medication delivery device 300 defines an internal space 19 to receive the medication cartridge 20. The medication cartridge 20 extends along a longitudinal direction between a distal end 21 and a proximate end 23. The medication cartridge 20 includes a vial 20a having a septum at the distal end 21. The medication cartridge 20 contains multiple doses of medication and includes a displacement member 22 disposed at least partially inside the vial 20a of the medication cartridge 20 between the proximate end 23 to the distal end 21. The displacement member 22 is movable along the longitudinal direction of the medication cartridge 20 from the proximate end 23 to the distal end 21 to deliver medication from the distal end 21 of the medication cartridge 20. The displacement member 22 is. In the embodiment depicted in FIG. 5A, the displacement member 22 is a plunger. The vial 20a of the medication cartridge 20 may have a transparent or semi-transparent wall such that a user can observe the medication inside the vial 20a. Dimension/dose marks can be provided on the vial wall. Exemplary insulin cartridges are commercially available from Novo Nordisk A/S (Bagsvaerd, Denmark) under the trade designation of NovoLog, and from Eli Lilly and Company (Indianapolis, Indiana) under the trade designation of HUMALOG.

The medication delivery device 300 further includes a drive mechanism 32 disposed at an end of the internal space 19, opposite the opening 15. The drive mechanism 32 mechanically connects to an advancing member 34 and configured to move the advancing member 34 toward the opening 15 in the longitudinal direction. In the embodiment depicted in FIG. 5A, the advancing member 34 is a piston. The piston 34 includes a piston rod 342 mechanically coupled with the drive mechanism 32 and a pressure block 344 to be engaged with the displacement member 22 (e.g., a plunger) of the medication cartridge 20 to advance the plunger 22 forward inside the medication cartridge 20 when the medication cartridge 20 is received in the housing 10 of the medication delivery device 300. It is to be understood that the advancing member 34 of the medication delivery device 300 may have any suitable configurations other than a piston.

The medication delivery device 300 further includes one or more location sensors 42 positioned to detect a location of the advancing member 34 or the displacement member 22 within the medication cartridge 20. For example, when the displacement member 22 (e.g., a plunger) is advanced by the advancing member 34 (e.g., a piston) at any point between the proximate end 23 and the distal end 21 along the longitudinal direction, the location sensors 42 can emit an optical signal toward the medication cartridge 20, and collect the reflected or transmitted optical signal from the medication cartridge 20. The location sensors 42 are mounted inside the housing 10, facing the medication cartridge 20. In some embodiments, an array of location sensors 42 are arranged along the longitudinal direction of the housing 10 between the proximate end 23 and the distal end 21 of the medication cartridge 20 to detect the location of the advancing member 34 or the displacement member 22 within the medication cartridge 20.

The medication delivery device 300 further includes a printed circuit board assembly (PCBA) 17 that may include various control circuitry or electronic components such as, for example, logic circuitry, drive controller circuitry, wireless communication circuitry, memory, etc. The printed circuit board assembly (PCBA) 17 is supported by the housing 10, powered by the battery 174, and functionally connected to other components of the medication delivery device 300 such as, for example, the drive mechanism 32, the skin sensor 16, the display 12, etc. The control circuitry is configured to execute one or more algorithms stored in the memory to determine an intended dose of medication to be delivered.

The control circuitry is further configured to obtain information from the one or more locations sensors 42, the information indicating the location of the advancing member 34 or the displacement member 22 within the medication cartridge 20. The control circuitry can execute one or more algorithms to determine a first position of the displacement member 22 within the medication cartridge 20 based on the information from the one or more location sensors 42. The first position identifies the location of the displacement member 22 before delivery of the dose of medication.

In some embodiments, the one or more locations sensors 42 can detect signals when a new medication cartridge is received by the housing 10. After receiving the signals from the locations sensors 42, the control circuitry can generate and present an instruction to the user to prime the medication delivery device. After the device is primed, the one or more locations sensors 42 can obtain information indicating the location of the advancing member 34 or the displacement member 22 within the medication cartridge 20 for the control circuitry to determine whether the device is properly primed. After determining that the device is properly primed, the control circuitry can determine the first position of the displacement member 22 before delivery of the dose of medication.

With the determined first position, the control circuitry can determine a second position of the advancing member 34 or the displacement member 22 within the medication device based on the intended dose of medication to be delivered. The second position indicates a desired location of the advancing member 34 or the displacement member 22 within the medication cartridge 20 after delivery of the dose of medication.

With the determined first and second positions, the control circuitry can instruct the drive mechanism 32 to advance the displacement member 22 from the determined first position, and instruct the drive mechanism 32 to cease advancing the displacement member 22 upon obtaining the information from the one or more location sensors 42 indicating that the advancing member 34 or the displacement member 22 has reached the second position.

After the delivery of the dose of medication, the control circuitry is further configured to determine a third position of the advancing member 34 or the displacement member 22 within the medication cartridge 20 after the displacement member 22 has ceased advancement based on the information from the one or more location sensors 42. The control circuitry is further configured to determine an actual dose of medication delivered based on the first position and the third position of the advancing member 34 or the displacement member 22 within the medication cartridge 20. The actual dose refers to the amount of medication being delivered out of the medication cartridge 20 after the displacement member 22 has ceased advancement. The control circuitry can compare the actual dose to the intended dose. When the actual dose is not the same as the intended dose, the control circuitry can execute one or more algorithms to calculate an insulin-on-board (IOB) value based on the actual dose, and calculate a subsequent intended dose for a next predetermined time based on the calculated IOB value.

### Automatically Delivering Medication to User

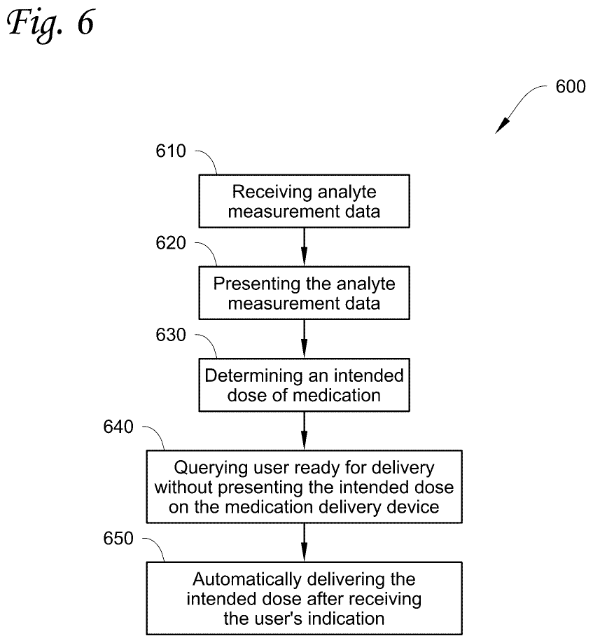

FIG. 6 is a flow diagram of a method 600 of delivering medication using a medication delivery device, according to one embodiment. The medication delivery device includes one or more user interface elements, control circuitry, and a memory. It is to be understood that the method 600 can be implemented by any medication delivery and management systems and/or any medication delivery devices described herein such as, for example, the medication delivery and management system 100 of the embodiment depicted in FIG. 1, and the medication delivery devices 200, 300 of the embodiments depicted in FIGS. 2-5B.

At 610, the medication delivery device receives analyte measurement data of a user. The medication delivery device can include a wireless communication component to receive analyte measurement data from an analyte sensor system such as, for example, the analyte sensor system 102 shown in FIG. 1. For example, in some embodiments, the medication delivery device may receive glucose data (e.g., a current glucose level) or glucose trend data (e.g., a rate of glucose level change) from a glucose sensor system. The method 600 then proceeds to 620.

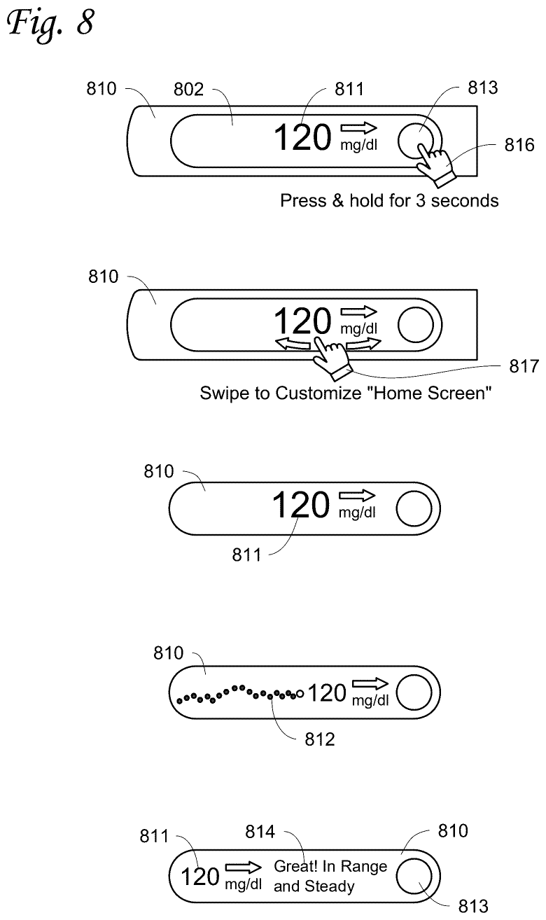

At 620, the medication delivery device presents, via the one or more user interface elements, information related to the analyte measurement data to the user. For example, in some embodiments, when the medication delivery device determines that the received glucose data or trend data are beyond predetermined criteria, the medication delivery device can generate alarms or alerts to present on the user interface elements to the user. In some embodiments, when the medication delivery device determines that the received glucose data or trend data are within the predetermined criteria, the medication delivery device can present the received glucose data and/or trend data on the user interface elements to the user without triggering the alarms or alerts. The method 600 then proceeds to 630. FIG. 8 shows an example user interface 810 for the medication delivery device that includes a graphical presentation of glucose data 811 and/or glucose trend data 812 on a display or touch screen 802 thereof. The trend data 812 include a series of glucose data along an axis indicating time. The user interface 810 further includes a virtual touch button 813 on the touch screen 802 to receive a user's input 816 (e.g., to push and hold by the user's finger) to display the data. The touch screen 802 can be configured to receive the user's input 817 (e.g., to swipe by the user's finger) to change the graphical presentation of the glucose data 811 and/or the glucose trend data 812. The user interface 810 further includes a graphical presentation of notification 814 regarding the displayed glucose data 811 and/or trend data 813.

At 630, the medication delivery device determines an intended dose of medication based on the received analyte measurement data. In some embodiments, the intended dose may be determined or adjusted further based on one or more therapeutic relevant conditions, one or more therapy parameters stored in the memory of the medication delivery device, or a calculated insulin-on-board (IOB) value. The method 600 then proceeds to 640.

The one or more therapeutic relevant conditions may include, for example, at least one of a meal size, a sleep plan, an exercise plan, etc. The one or more therapy parameters may include, for example, a daily dosage of long-acting insulin, dosages for different meal sizes or categories, an insulin sensitivity factor, a carbohydrate-to-insulin ratio, etc. In some embodiments, the medication delivery device can present, via the one or more user interface elements, a query to a user to solicit the user to input the one or more therapeutic relevant conditions.

In some embodiments, the content of the query and the timing of presenting the query to the user can be determined based on personalization pattern data of the user. The personalization pattern data may include the user's lifestyle factor data or historical data relating to, for example, user meal time in a day, user medication delivery time in a day, user exercise time in a day, user sleep time in a day, analyte measurement data (e.g., a glucose level) in response to the user's input of different therapeutic relevant conditions, actual dose data, etc. The lifestyle factor data or the historical data may be stored in memory of the medication delivery device. The medication delivery device can include an analyzing engine implemented by a control circuitry of the medication deliver device to analyze the user's lifestyle factor data or historical data to determine the content of the query and the timing (e.g., time-of-day) to present the query to a user on its user interface elements based on the user's personalization pattern data. It is to be understood that the analyzing engine can be implemented by any computing device/component of a medication delivery and management system such as 100 of the embodiment depicted in FIG. 1, to execute one or more algorithms to analyze the personalization pattern data and determine the query. Exemplary patient pattern data are described in U.S. Pat. Nos. 10,426,896, 11,083,852, 10,456,090, and PCT Publication No. WO/2021/072437, which are incorporated herein by reference.

The one or more therapy parameters may include, for example, a daily dosage of long-acting insulin, dosages for different meal sizes or categories, an insulin sensitivity factor, a carbohydrate-to-insulin ratio, etc. In some embodiments, the therapy parameters for a specific user may be stored in memory of the medication delivery device. The medication delivery device may receive, via a wireless communication component thereof, an update of the one or more therapy parameters from a mobile application such as, for example, the mobile application 104 of the embodiment depicted in FIG. 1.

In some embodiments, one or more algorithms can be executed by the mobile application via a secondary device such as the secondary device(s) 106 of the embodiment depicted in FIG. 1, or via a network such as the network 108 of FIG. 1 to update the therapy parameters. The mobile application can automatically deliver the updated therapy parameters to the medication delivery device. In some embodiments, the therapy parameters may be updated by a healthcare professional or manually by a user or a caregiver. In some embodiments, the medication delivery device may include an algorithm in memory to be executed by a processor to automatically update the therapy parameters. In some embodiments, the medication delivery device may receive, via the one or more user interface elements, a user's input to confirm the update of the one or more therapy parameters.

In some embodiments, a query may be presented, via a user interface of a secondary device (e.g., the secondary device(s) 106 in FIG. 1), to indicate whether a user needs to update the one or more therapeutic relevant conditions, the one or more therapy parameters, and/or manually adjust the intended dose determined by the medication delivery device.

At 640, the medication delivery device queries the user to indicate that the user is ready for the delivery without presenting the intended dose on any of the one or more user interface elements of the medication delivery device. The methods 600 then proceeds to 650.

In some embodiments, the medication delivery device may include one or more skin sensors positioned to determine a relative location of the medication delivery device with respect to the user's skin when the medication delivery device sits on the user's skin for medication delivery. The control circuitry of the medication delivery device may receive skin sensing data from skin sensors and execute one or more algorithms to determine whether the medication delivery device is at an acceptable location and orientation on the user's skin for medication delivery, and generate the corresponding queries to present on one or more of the user interface elements to indicate that the user is ready for the delivery or any adjustment is needed.

In some embodiments, the user may use a secondary device such as the secondary device(s) 106 of FIG. 1 to access and/or adjust the intended dose prior to querying the user at 640. After receiving the user input dose from the secondary device, the medication delivery device can present (e.g., display), via at least one of its user interface elements, an indication that the medication delivery device has received the user input dose from the secondary device and query the user to indicate the user is ready for the delivery of the user input dose.

At 650, the medication delivery device automatically delivers a dose of medication based on the intended dose determined at 630 after receiving the user's indication. When the user receives the indication from the user interface elements of the medication delivery device that it is ready for the medication delivery, the user can engage one user interface element such as a delivery button to trigger the device to deliver a dose of medication. It is to be understood that while the medication delivery device attempts to deliver a dose based on the intended dose automatically determined by the medication delivery device at 630, the actual dose delivered to the user may be different from intended dose for various reasons. Embodiments of medication delivery devices and methods are provided to control the device to deliver a dose based on the intended dose, determine the actual dose of medication after the delivery, calculate the insulin-on-board (IOB) value based on the actual dose, and determine a subsequent intended dose for a next predetermined time based on the calculated IOB value.

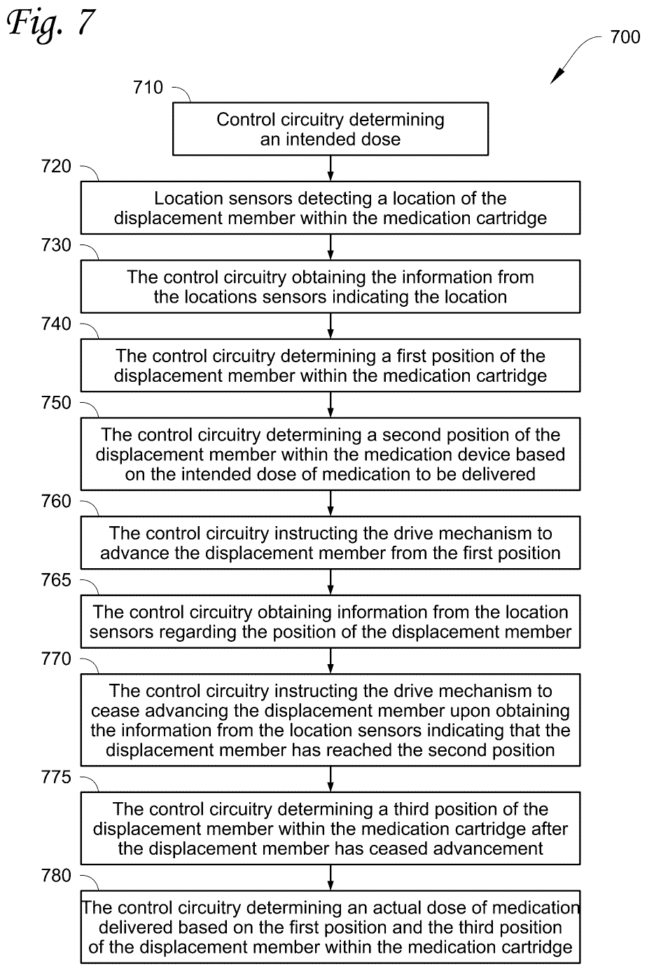

FIG. 7 is a flow diagram of a method 700 of delivering medication using a medication delivery device that includes a housing configured to receive a medication cartridge, according to another embodiment. A suitable medication cartridge extends between a distal end and a proximal end thereof and including a vial having a septum at the distal end and a displacement member inside the vial and movable from the proximal end to the distal end to deliver medication received by the vial from the distal end. A suitable medication delivery device used herein includes a drive mechanism inside the housing to advance the displacement member within the medication cartridge to deliver medication when the medication cartridge is received in the housing. The medication delivery device further includes one or more location sensors positioned to detect a location of the displacement member within the medication cartridge, and control circuitry configured to control operation of the medication delivery device. It is to be understood that the method 600 can be implemented by any medication delivery and management systems and/or any medication delivery devices described herein such as, for example, the medication delivery and management system 100 of FIG. 1, and the medication delivery devices 200, 300 of FIGS. 2-5B.

At 710, the control circuitry of the medication delivery device determines an intended dose of medication to be delivered. The intended dose of medication can be determined based on the received analyte measurement data. In some embodiments, the intended dose may be determined or adjusted further based on one or more therapeutic relevant conditions, one or more therapy parameters stored in the memory of the medication delivery device, or a calculated insulin-on-board (IOB) value. The control circuitry of the medication delivery device can determine the intended dose by any suitable methods described herein such as, for example, at 630 of the method 600 of FIG. 6.

At 720, the one or more location sensors of the medication delivery device detect a location of the displacement member within the medication cartridge and provide information indicating the location of the medication cartridge to the control circuitry. For example, in the embodiment depicted in FIG. 5A, the one or more location sensors 42 of the medication delivery device 300 are positioned to detect a location of the advancing member 34 or the displacement member 22 within the medication cartridge 20 and provide information indicating the location of the medication cartridge 20 to the control circuitry 17.

At 730, the control circuitry of the medication delivery device obtains the information from the one or more locations sensors indicating the location of the displacement member within the medication cartridge. For example, in the embodiment depicted in FIG. 5A, the control circuitry 17 of the medication delivery device 300 can obtain the information from the one or more locations sensors 42 indicating the location of the advancing member 34 or the displacement member 22 within the medication cartridge 20.

At 740, the control circuitry of the medication delivery device determines a first position of the displacement member within the medication cartridge based on the information from the one or more location sensors obtained at 730. The first position identifies the location of the displacement member before delivery of the dose of medication. For example, in the embodiment depicted in FIG. 5A, the control circuitry 17 of the medication delivery device 300 can execute one or more algorithms stored in the memory to determine a first position of the advancing member 34 or the displacement member 22 within the medication cartridge 20 based on the information from the one or more location sensors 42 obtained at 730.

At 750, the control circuitry of the medication delivery device determines a second position of the displacement member within the medication device based on the intended dose of medication to be delivered. The second position indicates a desired location of the displacement member within the medication cartridge after delivery of the dose of medication. For example, in the embodiment depicted in FIG. 5A, the control circuitry 17 of the medication delivery device 300 can execute one or more algorithms stored in the memory to determine a second position of the advancing member 34 or the displacement member 22 within the medication cartridge 20 based on the intended dose of medication to be delivered.

At 760, the control circuitry of the medication delivery device instructs the drive mechanism to advance the displacement member from the first position. For example, in the embodiment depicted in FIG. 5A, the control circuitry 17 of the medication delivery device 300 can instruct the drive mechanism 32 to advance the advancing member 34 or the displacement member 22 from the first position.

At 765, the control circuitry of the medication delivery device obtains information from the location sensors regarding the position of the displacement member. For example, in the embodiment depicted in FIG. 5A, when the drive mechanism 32 advances, via the advancing member 34, the displacement member 22, the control circuitry 17 of the medication delivery device 300 can obtain information from the location sensors 42 regarding the position of the advancing member 34 or the displacement member 22 within the medication cartridge 20.

At 770, the control circuitry of the medication delivery device instructs the drive mechanism to cease advancing the displacement member upon obtaining the information from the one or more location sensors indicating that the displacement member has reached the second position. For example, in the embodiment depicted in FIG. 5A, the control circuitry 17 of the medication delivery device 300 can instruct the drive mechanism 32 to cease advancing the displacement member 22 upon obtaining the information from the one or more location sensors 42 indicating that the advancing member 34 or the displacement member 22 has reached the second position.

At 775, after the delivery of the dose of medication, the control circuitry determines a third position of the displacement member within the medication cartridge after the displacement member has ceased advancement based on the information from the one or more location sensors. For example, in the embodiment depicted in FIG. 5A, the control circuitry 17 execute one or more algorithms stored in the memory to determine a third position of the advancing member 34 or the displacement member 22 within the medication cartridge 20 after the advancing member 34 or the displacement member 22 has ceased advancement based on the information from the one or more location sensors 42.

At 780, the control circuitry determines an actual dose of medication delivered based on the first position and the third position of the displacement member within the medication cartridge. For example, in the embodiment depicted in FIG. 5A, the control circuitry 17 execute one or more algorithms stored in the memory to determine an actual dose of medication delivered based on the first position and the third position of the advancing member 34 or the displacement member 22 within the medication cartridge 20. In some embodiments, the control circuitry can compare the actual dose to the intended dose. When the actual dose is not the same as the intended dose, the control circuitry can execute one or more algorithms to calculate an insulin-on-board (IOB) value based on the actual dose, and calculate a subsequent intended dose for a next predetermined time based on the calculated IOB value.

### Aspects

It is appreciated that any one of aspects can be combined with other aspect(s).
Aspect 1 is a method of delivering medication using a medication delivery device, the medication delivery device comprising one or more user interface elements, control circuitry, and a memory, the method comprising:
   receiving, via the medication delivery device, analyte measurement data of a user;
   presenting, via the one or more user interface elements, information related to the analyte measurement data to the user;
   determining an intended dose of medication based on the analyte measurement data;
   querying the user to indicate that the user is ready for delivering medication without presenting the intended dose on any of the one or more user interface elements of the medication delivery device; and
   automatically delivering the intended dose after receiving the user's indication.
Aspect 2 is the method of aspect 1, wherein the intended dose is determined further based on at least one of one or more therapeutic relevant conditions, one or more therapy parameters stored in the memory of the medication delivery device, and a calculated insulin-on-board (IOB) value.
Aspect 3 is the method of aspect 1 or 2, further comprising receiving, via a user interface of a secondary device, a user input dose representing a dose amount or an adjustment of the intended dose, wherein the secondary device is in wireless communication with the medication delivery device to transmit the received user input dose to the medication delivery device.
Aspect 4 is the method of aspect 3, further comprising displaying, via at least one of the one or more user interface elements of the medication delivery device, an indication that the medication delivery device has received the user input dose from the secondary device.
Aspect 5 is the method of aspect 4, further comprising querying the user to indicate the user is ready for the delivery of the user input dose, and automatically delivering the user input dose after receiving the user's indication.
Aspect 6 is the method of any one of aspects 3 to 5, further comprising querying the user, via the user interface of the secondary device, to indicate whether the user needs to manually adjust the intended dose.
Aspect 7 is the method of any one of aspects 1-6, further comprising presenting, via the one or more user interface elements, at a predetermined time, a query to a user to solicit the user to input one or more therapeutic relevant conditions.
Aspect 8 is the method of aspect 7, further comprising determining the query based on personalization pattern data of the user.
Aspect 9 is the method of any one of aspects 2-8, further comprising updating the one or more therapy parameters based on one or more previously stored therapy parameters and at least one of prior analyte measurement data, dose data, and meal data.
Aspect 10 is the method of aspect 9, further comprising receiving, via a wireless communication component of the medication delivery device, the update of the one or more therapy parameters from a mobile application.
Aspect 11 is the method of aspect 9 or 10, further comprising receiving, via the one or more user interface elements, a user's input to confirm the update of the one or more therapy parameters.
Aspect 12 is the method of any one of aspects 1-11, further comprising determining an actual dose of medication after delivering the intended dose.
Aspect 13 is the method of aspect 12, further comprising comparing the actual dose to the intended dose, when the actual dose is not the same as the intended dose, calculating an insulin-on-board (IOB) value based on the actual dose, and calculating a subsequent intended dose for a next predetermined time based on the calculated IOB value.
Aspect 14 is the method of any one of aspects 1-13, wherein querying the user to indicate that the user is ready comprises presenting an instruction to the user to prime the medication delivery device before automatically delivering the intended dose.
Aspect 15 is a medication delivery device comprising:
   a housing configured to receive a medication cartridge, the medication cartridge extending between a distal end and a proximal end thereof and including a vial having a septum at the distal end and a displacement member inside the vial and movable from the proximal end to the distal end to deliver medication received by the vial from the distal end;
   a drive mechanism inside the housing configured to advance the displacement member within the medication cartridge to deliver medication when the medication cartridge is received in the housing;
   one or more location sensors positioned to detect a location of the displacement member within the medication cartridge when the medication cartridge is received in the housing; and
   control circuitry configured to:
      determine an intended dose of medication to be delivered,
      obtain information from the one or more locations sensors, the information indicating the location of the displacement member within the medication cartridge,
      determine a first position of the displacement member within the medication cartridge based on the information from the one or more location sensors, wherein the first position identifies the location of the displacement member before delivery of the dose of medication,
      determine a second position of the displacement member within the medication device based on the intended dose of medication to be delivered, the second position indicating a desired location of the displacement member within the medication cartridge after delivery of the dose of medication,
      instruct the drive mechanism to advance the displacement member from the first position, and
      instruct the drive mechanism to cease advancing the displacement member upon obtaining the information from the one or more location sensors indicating that the displacement member has reached the second position.
Aspect 16 is the medication delivery device of aspect 15, wherein, after the delivery of the dose of medication, the control circuitry is configured to:
   determine a third position of the displacement member within the medication cartridge after the displacement member has ceased advancement based on the information from the one or more location sensors, and
   determine an actual dose of medication delivered based on the first position and the third position of the displacement member within the medication cartridge.
Aspect 17 is the medication delivery device of aspect 15 or 16, further comprising a push button to trigger the drive mechanism.
Aspect 18 is the medication delivery device of aspect 15, further comprising a skin sensor positioned at a distal end of the housing to detect skin.
Aspect 19 is the medication delivery device of aspect 15, further comprising a wireless communication component configured to receive analyte measurement data from an analyte sensor system.
Aspect 20. The medication delivery device of aspect 19, wherein the analyte measurement data include at least one of continuous glucose monitor (CGM) data, flash glucose monitor data, or glucose meter data.
Aspect 21 is the medication delivery device of aspect 19, wherein the wireless communication component is configured to receive one or more therapy parameters from a mobile application.
Aspect 22 is the medication delivery device of aspect 21, wherein the one or more therapy parameters include at least one of a daily dosage of long-acting insulin, dosages for different meal sizes, an insulin sensitivity factor, or a carbohydrate-to-insulin ratio.
Aspect 23 is the medication delivery device of aspect 15, further comprising one or more user interface elements to display analyte measurement data to a user.
Aspect 24 is the medication delivery device of aspect 15, further comprising one or more user interface elements to present a query to solicit the user to input one or more therapeutic relevant conditions.
Aspect 25 is the medication delivery device of aspect 24, wherein the one or more therapeutic relevant conditions include at least one of a meal size, or an exercise plan.
Aspect 26 is the medication delivery device of aspect 15, which is a smart insulin injection pen.
Aspect 27 is a method of delivering medication using a medication delivery device that includes a housing configured to receive a medication cartridge, the medication cartridge extending between a distal end and a proximal end thereof and including a vial having a septum at the distal end and a displacement member inside the vial and movable from the proximal end to the distal end to deliver medication received by the vial from the distal end, the medication delivery device further including a drive mechanism inside the housing configured to advance the displacement member within the medication cartridge to deliver medication when the medication cartridge is received in the housing, one or more location sensors positioned to detect a location of the displacement member within the medication cartridge, and control circuitry configured to control operation of the medication delivery device, the method comprising:
   the control circuitry determining an intended dose of medication to be delivered;
   the one or more location sensors detecting a location of the displacement member within the medication cartridge and providing information indicating the location of the medication cartridge to the control circuitry;
   the control circuitry obtaining the information from the one or more locations sensors indicating the location of the displacement member within the medication cartridge;
   the control circuitry determining a first position of the displacement member within the medication cartridge based on the information from the one or more location sensors, wherein the first position identifies the location of the displacement member before delivery of the dose of medication;
   the control circuitry determining a second position of the displacement member within the medication device based on the intended dose of medication to be delivered, the second position indicating a desired location of the displacement member within the medication cartridge after delivery of the dose of medication;
   the control circuitry instructing the drive mechanism to advance the displacement member from the first position; and
   the control circuitry instructing the drive mechanism to cease advancing the displacement member upon obtaining the information from the one or more location sensors indicating that the displacement member has reached the second position.
Aspect 28 is the method of aspect 27, further comprising
   after the delivery of the dose of medication, the control circuitry determining a third position of the displacement member within the medication cartridge after the displacement member has ceased advancement based on the information from the one or more location sensors; and
   the control circuitry determining an actual dose of medication delivered based on the first position and the third position of the displacement member within the medication cartridge.
Aspect 29 is the method of aspect 28, further comprising comparing the actual dose to the intended dose, when the actual dose is not the same as the intended dose, calculating an insulin-on-board (IOB) value based on the actual dose, and calculating a subsequent intended dose for a next predetermined time based on the calculated IOB value.
Aspect 30 is the method of aspect 27, further comprising receiving, via a wireless communication component, analyte measurement data from an analyte sensor system.
Aspect 31 is the method of aspect 30, wherein the analyte measurement data include at least one of continuous glucose monitor (CGM) data, flash glucose monitor data, or glucose meter data.
Aspect 32 is the method of aspect 27, further comprising receiving, via a wireless communication component, one or more therapy parameters from a mobile application.
Aspect 33 is the method of aspect 32, wherein the one or more therapy parameters include at least one of a daily dosage of long-acting insulin, dosages for different meal sizes, an insulin sensitivity factor, or a carbohydrate-to-insulin ratio.
Aspect 34 is the method of aspect 27, further comprising presenting, via a user interface, a query to the user to solicit the user to input one or more therapeutic relevant conditions.
Aspect 35 is the method of aspect 34, wherein the one or more therapeutic relevant conditions include at least one of a meal size, or an exercise plan.
Aspect 36 is the method of aspect 27, further comprising displaying, via a user interface of the medication delivery device, analyte measurement data to a user.
Aspect 37 is the method of aspect 36, further comprising displaying, via the user interface, an alert for a glucose measurement value out of a predetermined range.
Aspect 38 is the method of aspect 27, further comprising detecting a user's skin, via a skin sensor, to determine a relative location of a distal end of the device with respect to the user's skin.
Aspect 39 is the method of aspect 27, wherein the intended dose of medication is determined based on at least one of analyte measurement data, optionally, one or more therapeutic relevant conditions, one or more therapy parameters stored in the memory of the medication delivery device, or a calculated insulin-on-board (IOB) value.
Aspect 40 is the method of aspect 27, further comprising receiving, via a user interface, an input from a user indicating that the user is ready for the delivery.

The terminology used in this specification is intended to describe particular embodiments and is not intended to be limiting. The terms “a,” “an,” and “the” include the plural forms as well, unless clearly indicated otherwise. The terms “comprises” and/or “comprising,” when used in this specification, specify the presence of the stated features, integers, steps, operations, elements, and/or components, but do not preclude the presence or addition of one or more other features, integers, steps, operations, elements, and/or components.

With regard to the preceding description, it is to be understood that changes may be made in detail, especially in matters of the construction materials employed and the shape, size, and arrangement of parts without departing from the scope of the present disclosure. This specification and the embodiments described are exemplary only, with the true scope and spirit of the disclosure being indicated by the claims that follow.

1-40. (canceled)

41. A method of delivering medication using a medication delivery device, the medication delivery device comprising one or more user interface elements, control circuitry, and a memory, the method comprising:
receiving, via the medication delivery device, analyte measurement data of a user;
presenting, via the one or more user interface elements, information related to the analyte measurement data to the user;
determining an intended dose of medication based on the analyte measurement data;
querying the user to indicate that the user is ready for delivering medication without presenting the intended dose on any of the one or more user interface elements of the medication delivery device; and
automatically delivering the intended dose after receiving the user's indication.

42. The method of claim 41, wherein the intended dose is determined further based on at least one of one or more therapeutic relevant conditions, one or more therapy parameters stored in the memory of the medication delivery device, and a calculated insulin-on-board (IOB) value.

43. The method of claim 41, further comprising:
receiving, via a user interface of a secondary device, a user input dose representing a dose amount or an adjustment of the intended dose, wherein the secondary device is in wireless communication with the medication delivery device to transmit the received user input dose to the medication delivery device;
querying the user to indicate the user is ready for the delivery of the user input dose; and
automatically delivering the user input dose after receiving the user's indication.

44. The method of claim 41, further comprising:
presenting, via the one or more user interface elements, at a predetermined time, a query to a user to solicit the user to input one or more therapeutic relevant conditions; and
determining the query based on personalization pattern data of the user.

45. The method of claim 42, further comprising updating the one or more therapy parameters based on one or more previously stored therapy parameters and at least one of prior analyte measurement data, dose data, and meal data.

46. The method of claim 45, further comprising:
receiving, via a wireless communication component of the medication delivery device, the update of the one or more therapy parameters from a mobile application; and
receiving, via the one or more user interface elements, a user's input to confirm the update of the one or more therapy parameters.

47. The method of claim 41, further comprising determining an actual dose of medication after delivering the intended dose.

48. The method of claim 47, further comprising comparing the actual dose to the intended dose, when the actual dose is not the same as the intended dose, calculating an insulin-on-board (IOB) value based on the actual dose, and calculating a subsequent intended dose for a next predetermined time based on the calculated IOB value.

49. The method of claim 41, wherein querying the user to indicate that the user is ready comprises presenting an instruction to the user to prime the medication delivery device before automatically delivering the intended dose.

50. A medication delivery device comprising:
a housing configured to receive a medication cartridge, the medication cartridge extending between a distal end and a proximal end thereof and including a vial having a septum at the distal end and a displacement member inside the vial and movable from the proximal end to the distal end to deliver medication received by the vial from the distal end;
a drive mechanism inside the housing configured to advance the displacement member within the medication cartridge to deliver medication when the medication cartridge is received in the housing;
one or more location sensors positioned to detect a location of the displacement member within the medication cartridge when the medication cartridge is received in the housing; and
control circuitry configured to:
determine an intended dose of medication to be delivered,
obtain information from the one or more locations sensors, the information indicating the location of the displacement member within the medication cartridge,
determine a first position of the displacement member within the medication cartridge based on the information from the one or more location sensors, wherein the first position identifies the location of the displacement member before delivery of the dose of medication,
determine a second position of the displacement member within the medication device based on the intended dose of medication to be delivered, the second position indicating a desired location of the displacement member within the medication cartridge after delivery of the dose of medication,
instruct the drive mechanism to advance the displacement member from the first position, and
instruct the drive mechanism to cease advancing the displacement member upon obtaining the information from the one or more location sensors indicating that the displacement member has reached the second position.

51. The medication delivery device of claim 50, wherein, after the delivery of the dose of medication, the control circuitry is configured to:
determine a third position of the displacement member within the medication cartridge after the displacement member has ceased advancement based on the information from the one or more location sensors, and
determine an actual dose of medication delivered based on the first position and the third position of the displacement member within the medication cartridge.

52. The medication delivery device of claim 50, further comprising a push button to trigger the drive mechanism.

53. The medication delivery device of claim 50, further comprising a skin sensor positioned at a distal end of the housing to detect skin.

54. The medication delivery device of claim 50, further comprising a wireless communication component configured to receive analyte measurement data from an analyte sensor system, wherein the analyte measurement data include at least one of continuous glucose monitor (CGM) data, flash glucose monitor data, or glucose meter data.

55. The medication delivery device of claim 54, wherein the wireless communication component is configured to receive one or more therapy parameters from a mobile application, and
wherein the one or more therapy parameters include at least one of a daily dosage of long-acting insulin, dosages for different meal sizes, an insulin sensitivity factor, or a carbohydrate-to-insulin ratio.

56. The medication delivery device of claim 50, further comprising one or more user interface elements to:
display analyte measurement data to a user, and
present a query to solicit the user to input one or more therapeutic relevant conditions, wherein the one or more therapeutic relevant conditions include at least one of a meal size, or an exercise plan.

57. A method of delivering medication using a medication delivery device that includes a housing configured to receive a medication cartridge, the medication cartridge extending between a distal end and a proximal end thereof and including a vial having a septum at the distal end and a displacement member inside the vial and movable from the proximal end to the distal end to deliver medication received by the vial from the distal end, the medication delivery device further including a drive mechanism inside the housing configured to advance the displacement member within the medication cartridge to deliver medication when the medication cartridge is received in the housing, one or more location sensors positioned to detect a location of the displacement member within the medication cartridge, and control circuitry configured to control operation of the medication delivery device, the method comprising:
the control circuitry determining an intended dose of medication to be delivered;
the one or more location sensors detecting a location of the displacement member within the medication cartridge and providing information indicating the location of the medication cartridge to the control circuitry;
the control circuitry obtaining the information from the one or more locations sensors indicating the location of the displacement member within the medication cartridge;
the control circuitry determining a first position of the displacement member within the medication cartridge based on the information from the one or more location sensors, wherein the first position identifies the location of the displacement member before delivery of the dose of medication;
the control circuitry determining a second position of the displacement member within the medication device based on the intended dose of medication to be delivered, the second position indicating a desired location of the displacement member within the medication cartridge after delivery of the dose of medication;
the control circuitry instructing the drive mechanism to advance the displacement member from the first position; and
the control circuitry instructing the drive mechanism to cease advancing the displacement member upon obtaining the information from the one or more location sensors indicating that the displacement member has reached the second position.

58. The method of claim 57, further comprising
after the delivery of the dose of medication, the control circuitry determining a third position of the displacement member within the medication cartridge after the displacement member has ceased advancement based on the information from the one or more location sensors; and
the control circuitry determining an actual dose of medication delivered based on the first position and the third position of the displacement member within the medication cartridge.

59. The method of claim 58, further comprising comparing the actual dose to the intended dose, when the actual dose is not the same as the intended dose, calculating an insulin-on-board (IOB) value based on the actual dose, and calculating a subsequent intended dose for a next predetermined time based on the calculated IOB value.

60. The method of claim 57, wherein the intended dose of medication is determined based on at least one of analyte measurement data, optionally, one or more therapeutic relevant conditions, one or more therapy parameters stored in the memory of the medication delivery device, or a calculated insulin-on-board (IOB) value.
